(19) United States
(12) Patent Application Publication
(10) Pub. No.: US 2020/0069729 A1
(43) Pub. Date: Mar. 5, 2020

(54) Combination Therapy for Treating Proliferative Diseases

(71) Applicant: BIONOMICS LIMITED, Thebarton (AU)

(72) Inventors: Gabriel Kremmidiotis, Flagstaff Hill (AU); Annabell Leske, Allenby Gardens (AU); Tina Lavranos, Colonel Light Gardens (AU)

(21) Appl. No.: 16/679,193

(22) Filed: Nov. 9, 2019

Related U.S. Application Data
(63) Continuation of application No. 15/900,254, filed on Feb. 20, 2018.
Application No. 15/900,254 is a continuation of application No. 14/279,520, filed on May 16, 2014, now abandoned.
Application No. 14/279,520 is a continuation of application No. 12/954,154, filed on Nov. 24, 2010, now abandoned.
(60) Provisional application No. 61/264,749, filed on Nov. 27, 2009.

(51) Int. Cl. A61K 33/24; A61K 31/7068; A61K 31/282; A61K 45/06; A61K 31/665; A61K 31/343

(52) U.S. Cl. CPC A61K 33/24; A61K 31/7068; A61K 31/343; A61K 45/06; A61K 31/665; A61K 31/282

(57) ABSTRACT

The present invention relates generally to new chemical combinations and methods for their use in the treatment of proliferative diseases and in particular cancer.

### RELATED APPLICATIONS

This application claims priority to U.S. patent application Ser. No. 12/954,154 filed Nov. 24, 2010 which claims priority to U.S. Provisional Patent Application No. 61/264,749 filed Nov. 27, 2009, which are hereby incorporated herein by reference.

### FIELD OF THE INVENTION

The present invention relates generally to new chemical combinations and methods for their use in the treatment of proliferative diseases and in particular cancer.

### BACKGROUND OF THE INVENTION

Cancer is typically treated with either chemotherapy and/or radiation therapy. While often effective to destroy a significant amount of tumour cells, such therapies often leave behind a number of tumour cells that are resistant to the treatment. These resistant cells can proliferate to form new tumours that are then resistant to treatment. As a result, the constant use of known combinations of chemotherapeutic drugs has given rise to multidrug resistant (‘MDR’) tumour cells.

The mode of proliferative diseases, such as tumours, is multi-factorial. For instance, research over the last forty years has led to the realisation that cytotoxic agents (or anti-proliferative agents) includes anti-metabolic agents which intefere with microtubule formulation, alkylating agents which are able to cross-link DNA, platinum based agents which are able to interfere with DNA alkylation by blocking DNA replication, antitumour antibiotic agents, topoisomerase inhibitors, etc. In the treatment of such diseases drugs with different mechanisms may be combined (i.e, combination therapies) with beneficial effects including the effective treatment of MDR tumour cells and to minimise side effects such as undesireable cytotoxicity. The difficulty here is though that not all known antiproliferative agents provide useful or beneficial effects in combination and accordingly research in many laboratories is presently focused on developing new and useful anti-proliferative combination partners.

It has now been found that a combination comprising a compound selected from a small class of particularly substituted benzofuran tubulin polymerisation inhibitors and at least one other anti-proliferative agent selected from a group of specific agents is particularly useful in treating proliferative diseases and in particular cancer.

### SUMMARY OF THE INVENTION

The present invention provides a pharmaceutical combination for treating a proliferative disease comprising: (a) a compound of formula (I) or a salt, solvate or prodrug thereof;

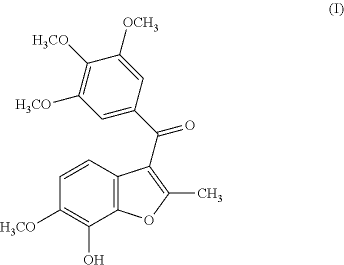 [SMILES: COc1cc(C(=O)c2c(C)oc3c(O)c(OC)ccc23)cc(OC)c1OC]

and (b) at least one other anti-proliferative agent selected from alkylating agents, antitumour antibiotics, antimetabolites, natural alkaloids and inhibitors of protein tyrosine kinases and/or serine/threonine kinases, for simultaneous, separate or sequential administration.

The present invention also provides a method for treating a proliferative disease including the step of administering to a patient in need thereof: (a) a compound of formula (I) or a pharmaceutically acceptable salt, solvate or prodrug thereof;

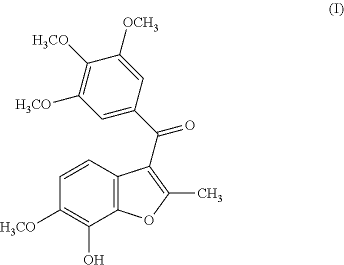 [SMILES: COc1cc(C(=O)c2c(C)oc3c(O)c(OC)ccc23)cc(OC)c1OC]

and (b) at least one other anti-proliferative agent selected from alkylating agents, antitumour antibiotics, antimetabolites, natural alkaloids and inhibitors of protein tyrosine kinases and/or serine/threonine kinases, simultaneously, separately, or sequentially.

The present invention further provides a pharmaceutical composition comprising (a) a compound of formula (I) or a pharmaceutically acceptable salt, solvate or prodrug thereof:

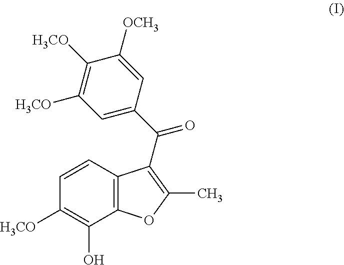 [SMILES: COc1cc(C(=O)c2c(C)oc3c(O)c(OC)ccc23)cc(OC)c1OC]

and (b) at least one other anti-proliferative agent selected from alkylating agents, antitumour antibiotics, antimetabolites, natural alkaloids and inhibitors of protein tyrosine kinases and/or serine/threonine kinases.

The present invention further provides a kit comprising:
(a) an amount of a compound of formula (I) or a pharmaceutically acceptable salt, solvate or prodrug thereof:

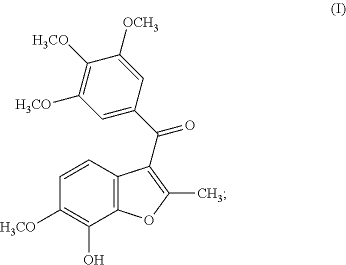 [SMILES: COc1cc(C(=O)c2c(C)oc3c(O)c(OC)ccc23)cc(OC)c1OC]

(b) an amount of at least one other anti-proliferative agent selected from alkylating agents, antitumour antibiotics, antimetabolites, natural alkaloids and inhibitors of protein tyrosine kinases and/or serine/threonine kinases; and
(c) instructions for use of (a) and (b) in combination for treating a proliferative disease.

Unexpectedly it has been found that the effects in treating proliferative diseases with a combination which comprises: (a) a compound of formula (I) or a pharmaceutically acceptable salt, solvate or prodrug thereof:

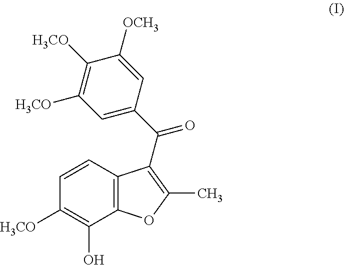 [SMILES: COc1cc(C(=O)c2c(C)oc3c(O)c(OC)ccc23)cc(OC)c1OC]

and (b) at least one other anti-proliferative agent selected from alkylating agents, antitumour antibiotics, antimetabolites, natural alkaloids and inhibitors of protein tyrosine kinases and/or serine/threonine kinases, are greater than that can be achieved with either (a) or (b) alone. That is, the present combinations have been found to possess beneficial additive (or sometimes synergistic) effects in anti-cancer therapy.

### BRIEF DESCRIPTION OF THE FIGURES

FIG. 1—Table of combination index values. Range of combination index=synergy (<0.1-0.90), additive (0.90-1.10), and no additive benefit (antagonism) (1.10->10).

FIG. 2—Graph showing tumour growth over 23 days calculated relative to average tumour volume (mm³). [symbol] denotes cisplatin treatment day. [symbol] denotes Example 2 treatment day.

FIG. 3—Graph showing % survival of mice vs days of treatment.

FIG. 4—Graph showing tumour growth ratio (Day X/Day 1) vs days of treatment for combination with Doxorubicin or 5-Fluorouracil.

FIG. 5—depicts a graph of % perfusion control against an amount of compound (mg/kg) in relation to comparative levels of vascular shutdown (reduction in tumour perfusion) between CA4P and compound Example 2 of the present invention.

FIG. 6—depicts a graph of Tumour Volume ratio (Day*/Day 1) against time (Days) in relation to tumour growth inhibition of compound example 2 in Balb/c nu/nu mice bearing MDA-MB-231 orthotopic breast solid tumours.

FIG. 7—plots of Tumor Volume (mm³) as a function of time (days) for saline (A), Example 2 (B), Cisplatin (C), and Example 2+Cisplatin (D).

FIG. 8—graph showing % survival of mice bearing calcu-6 xenografts treated with Example 2 and Cisplatin as a function of time (days).

FIG. 9—plots of Tumor Volume (mm³) as a function of time (days) for saline (A), Example 2 (B), Gemcitabine (C), and Example 2+Gemcitabine (D).

FIG. 10—graph showing % survival of mice bearing Calcu-6 xenografts treated with Example 2 and Gemcitabine, as a function of time (days).

FIG. 11—plots of % weight loss from day 1 as a function of time (days) for saline (A), Example 2 (B), Gemcitabine (C), and Example 2+Gemcitabine (D).

FIG. 12—plots of % weight loss from day 1 as a function of time (days) for saline (A), Example 2 (B), Cisplatin (C) and Example 2+Cisplatin (D).

### DESCRIPTION OF THE PREFERRED EMBODIMENTS

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### Combination Partner (a); Compound of Formula (I)

Compound of formula (I) (2-Methyl-7-hydroxy-3-(3,4,5-trimethoxybenzoyl)-6-methoxybenzofuran) can be prepared by the synthetic methodology described in PCT/AU2007/000101 (WO 07/087684).

The compound of formula (I) is observed to be potent tubulin polymerisation inhibitor (TPI). TPI compounds are important in the treatment of cancers primarily as a result of their capacity to selectively shut down blood flow through a tumour. Compounds that inhibit tumour blood flow are generally referred to as vascular disrupting agents (VDAs) (Tozer, G. M.; Kanthou, C.; Baguley, B. C. Nature Rev., Vol. 5, 2005, 423). TPIs are VDAs because they inhibit a certain cell signalling pathway associated with microtubules, leading to interference in the regulation of the cytoskeleton of the endothelial cells that line the blood vessels of the tumour. As a result, these usually flat cells become more rounded, ultimately occluding blood flow through the vessels. The selectivity associated with these agents results from the fact that tumour vasculature is weaker and more prone to collapse than normal vasculature. Nonetheless, a number of the dose limiting toxicities associated with VDAs are due to a reduction in blood flow in healthy tissues. An important aspect of the compound of formula (I) is the combination of the specific C-6 and C-7 substituents together with the C-2 methyl group which appears to confer greater potency and selectivity when compared to other structurally related TPI compounds. In this compound selectivity is not simply reliant on the predisposition of tumour vasculature towards collapse when challenged with the VDA but on a capacity of the VDA to distinguish between tumour endothelial cells and normal endothelial cells. Normal endothelial cells, found in healthy tissues, are in a “quiescent” state and tumour endothelial cells are in an “activated” state. Most VDAs do not distinguish between these two states, for example, Combretastatin A4 is equally potent against quiescent and activated endothelial cells. However, the compound of formula (I) shows high potency towards tumour endothelial cells (activated) over normal endothelial cells (quiescent).

It will be appreciated that the compound of formula (I) can be administered to a subject as a pharmaceutically acceptable salt thereof. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

It will also be appreciated that any compound that is a prodrug of the compound of formula (I) is also within the scope and spirit of the invention. The term “pro-drug” is used in its broadest sense and encompasses those derivatives that are converted in vivo to the compound of formula (I). Such derivatives would readily occur to those skilled in the art, and include, for example, compounds where the free hydroxy group at the C-7 position is converted into an ester, such as an acetate or phosphate ester. Procedures for esterifying, eg. acylating, the compound of formula (I) are well known in the art and may include treatment of the compound with an appropriate carboxylic acid, anhydride or chloride in the presence of a suitable catalyst or base. A particularly preferred prodrug is a disodium phosphate ester. The disodium phosphate ester of the compound of the invention may be useful in increasing the solubility of the compound. This, for instance, may allow for delivery of the compound in a benign vehicle like saline. The disodium phosphate ester may be prepared in accordance with the methodology described in Pettit, G. R., et al, Anticancer Drug Des., 1995, 10, 299. Other texts which generally describe prodrugs (and the preparation thereof) include: Design of Prodrugs, 1985, H. Bundgaard (Elsevier); The Practice of Medicinal Chemistry, 1996, Camille G. Wermuth et al., Chapter 31 (Academic Press); and A Textbook of Drug Design and Development, 1991, Bundgaard et al., Chapter 5, (Harwood Academic Publishers).

The compound of formula (I) (or a prodrug thereof) may be in crystalline form either as the free compound or as a solvate (e.g. hydrate) and it is intended that both forms are within the scope of the present invention. Methods of solvation are generally known within the art.

### Combination Partner (b); the Anti-Proliferative Agent

The “at least one other anti-proliferative agent” or combination partner (b) may include any chemotherapeutic agent that can be used in the treatment of proliferative diseases selected from known anti-proliferative alkylating agents, antitumour antibiotics, antimetabolites, natural alkaloids and inhibitors of protein tyrosine kinases and/or serine/threonine kinases. Said combination partners being suitable in respect to providing at least the beneficial additive effects outlined more specifically herein. Such agents include:
(i) alkylating agents, such as cis-platinum(II)-diaminedichloride (platinol or cisplatin); oxaliplatin (Eloxatin or Oxaliplatin Medac); and carboplatin (Paraplatin);
(ii) antitumour antibiotics, including those selected from the group comprising anthracyclines, such as doxorubicin (Adriamycin, Rubex);
(iii) antimetabolites, including folic acid analogues such as pyrimidine analogues such as 5-fluorouracil (Fluoruracil, 5-FU), gemcitabine (Gemzar), or histone deacetylase inhibitors (HDI) for instance, Vorinostat (rINN);
(iv) natural alkaloids, including paclitaxel (Taxol);
(v) inhibitors of protein tyrosine kinases and/or serine/threonine kinases including Sorafenib (Nexavar), Erlotinib (Tarceva), Dasatanib (BMS-354825 or Sprycel).

Preferred anti-proliferative agents include:

carboplatin, cisplatin, doxorubicin, 5-FU, gemcitabine, paclitaxel, oxaliplatin, sorafenib, tarceva, dasatanib, and vorinostat.

These above anti-proliferative agents are known in the art and their synthesis would also be known to those skilled in the art.

As used herein the term “proliferative disease” broadly encompasses any neoplastic disease including those which are potentially malignant (pre-cancerous) or malignant (cancerous). The term therefore encompasses the treatment of tumours.

Accordingly, the term “tumour” is used generally to define any malignant cancerous or pre-cancerous cell growth, and may include leukemias, and carcinomas such as melanomas, colon, lung, ovarian, skin, breast, pancrease, pharynx, brain prostate, CNS, and renal cancers, as well as other cancers.

In a preferred embodiment the combination may be used in the treatment of tumours and in particular in the following tumours: breast carcinoma, brain glioblastoma, colorectal carcinoma, lung carcinoma, ovary carcinoma, pancreas carcinoma, prostate carcinoma, renal cell carcinoma, and pharynx squamous cell carcinoma.

Thus the present invention also provides a method of treating tumours comprising the administration of an effective amount of (a) a compound of formula (I) or a pharmaceutically acceptable salt, solvate or prodrug thereof and (b) at least one other anti-proliferative agent.

An “effective amount” is intended to mean that the amount of each combination partner, when administered to a mammal (in particular a human) in need of such treatment, is sufficient to effect treatment for a particular proliferative disease. Thus, for example, a therapeutically effective amount of a compound of the formula (I) (or a pharmaceutically acceptable salt, solvate, or prodrug thereof) is a quantity sufficient to potentiate the activity of the at least one anti-proliferative agent such that a targeted disease state is reduced or alleviated.

Accordingly, the terms “treatment” or “treating” as used herein are intended to include at least partially attaining the desired effect, or delaying the onset of, or inhibiting the progression of, or halting or reversing altogether the onset or progression of the particular disease (e.g., tumour) being treated.

Without wanting to be bound by any particular theory it is believed that the tumour vascular disruption effect caused by compound of formula (I) is transient (at least in some tumours) with tumour re-vascularisation occurring after around 48 hrs. It is believed that the synergistic or additive effect proposed by the present combination is (at least in respect of some of the combination partners for (a)) the inhibition or delay of this re-vascularisation event and tumour recovery from the initial disruption with combination partner (a).

In some embodiments the following combinations of (a) and (b) have found to be particularly beneficial in the treatment of particular tumours:

| | (a) | + | (b) | Treatment of |
|---|---|---|---|---|
| 1. | Compound of formula (I) or | + | carboplatin | breast carcinoma |
| | prodrug thereof | | | |
| 2. | Compound of formula (I) or | + | carboplatin | pancrease carcinoma |
| | prodrug thereof | | | |
| 3. | Compound of formula (I) or | + | carboplatin | renal cell carcinoma |
| | prodrug thereof | | | |
| 4. | Compound of formula (I) or | + | cisplatin | breast carcinoma |
| | prodrug thereof | | | |
| 5. | Compound of formula (I) or | + | cisplatin | brain glioblastoma |
| | prodrug thereof | | | |
| 6. | Compound of formula (I) or | + | cisplatin | colorectal carcinoma |
| | prodrug thereof | | | |
| 7. | Compound of formula (I) or | + | cisplatin | lung carcinoma |
| | prodrug thereof | | | |
| 8. | Compound of formula (I) or | + | cisplatin | pancrease carcinoma |
| | prodrug thereof | | | |
| 9. | Compound of formula (I) or | + | doxorubicin | colorectal carcinoma |
| | prodrug thereof | | | |
| 10. | Compound of formula (I) or | + | doxorubicin | pancrease carcinoma |
| | prodrug thereof | | | |
| 11. | Compound of formula (I) or | + | 5-FU | breast carcinoma |
| | prodrug thereof | | | |
| 12. | Compound of formula (I) or | + | 5-FU | brain glioblastoma |
| | prodrug thereof | | | |
| 13. | Compound of formula (I) or | + | 5-FU | colorectal carcinoma |
| | prodrug thereof | | | |
| 14. | Compound of formula (I) or | + | 5-FU | lung carcinoma |
| | prodrug thereof | | | |
| 15. | Compound of formula (I) or | + | 5-FU | ovary carcinoma |
| | prodrug thereof | | | |
| 16. | Compound of formula (I) or | + | 5-FU | pancrease carcinoma |
| | prodrug thereof | | | |
| 17. | Compound of formula (I) or | + | 5-FU | prostate carcinoma |
| | prodrug thereof | | | |
| 18. | Compound of formula (I) or | + | 5-FU | renal cell carcinoma |
| | prodrug thereof | | | |
| 19. | Compound of formula (I) or | + | gemcitabine | lung carcinoma |
| | prodrug thereof | | | |
| 20. | Compound of formula (I) or | + | oxaliplatin | colorectal carcinoma |
| | prodrug thereof | | | |
| 21. | Compound of formula (I) or | + | sorafenib | brain glioblastoma |
| | prodrug thereof | | | |
| 22. | Compound of formula (I) or | + | tarceva | colorectal carcinoma |
| | prodrug thereof | | | |
| 23. | Compound of formula (I) or | + | tarceva | pharynx squamous |
| | prodrug thereof | | | cell carcinoma |
| 24. | Compound of formula (I) or | + | tarceva | prostate carcinoma |
| | prodrug thereof | | | |
| 25. | Compound of formula (I) or | + | dasatanib | colorectal carcinoma |
| | prodrug thereof | | | |
| 26. | Compound of formula (I) or | + | dasatanib | ovary carcinoma |
| | prodrug thereof | | | |
| 27. | Compound of formula (I) or | + | dasatanib | pharynx squamous |
| | prodrug thereof | | | cell carcinoma |
| 28. | Compound of formula (I) or | + | dasatanib | prostate carcinoma |
| | prodrug thereof | | | |
| 29. | Compound of formula (I) or | + | vorinostat | prostate carcinoma |
| | prodrug thereof | | | |
| 30. | Compound of formula (I) or | + | vorinostat | lung carcinoma |
| | prodrug thereof | | | |

Clinical studies such as open-label, dose escalation studies in patients with proliferative diseases may include studies to prove the synergism or additive effects of the active ingredients of the combination as opposed to providing no additional benefit (which may include increased toxicity or the lowering of the potency (or effectiveness) of one of the combination partners. The beneficial effects can be determined directly through the results of these studies which are known as such to a person skilled in the art. These studies are also able to compare the effects of a monotherapy using the active ingredients and a combination of the invention. Preferably, the dose of combination partner (a) may be escalated until the Maximum Tolerated Dosage (MTD) is reached, and agent (b) is administered as a fixed dose. Alternatively, combination partner (a) is administered in a fixed dose and the dose of agent (b) is escalated. Each patient may receive doses of agent (a) either daily or intermittent. The efficacy of the treatment can be determined in such studies, e.g., after 6, 12, 18 or 24 weeks by evaluation of symptom scores every 6 weeks. In another study agent (b) would be given at recommended dose and combination partner (a) progressively escalated from 4.2, 8.4, 12.6 and 16.0 mg/m². If no dose limiting toxicities were identified agent (a) could be used at the highest dose in a further clinical study.

The administration of the pharmaceutical combination of the present invention may result not only in a beneficial effect, e.g., a synergistic or additive therapeutic effect, for instance, with regard to alleviating, delaying progression of or inhibiting the symptoms, but also in further surprising beneficial effects. Such other effects may include fewer side effects, an improved quality of life or a decreased morbidity, compared with a monotherapy applying only one of the pharmaceutically active ingredients used in the combination of the present invention.

A further benefit of the invention is that lower doses of the active ingredients of the combination can be used. The dosages need not only be smaller but may also be applied less frequently, which may diminish the incidence or severity of side effects.

The term “administration” relates to the co-administration of the combination partners to a single patient, and is intended to include treatment regimens in which the agents are not necessarily administered by the same route of administration or at the same time. Accordingly, combination partners (a) and (b) may be administered together, one after the other or separately in one combined unit dosage form or in two separate unit dosage forms. The unit dosage form may also be a fixed combination such as a pharmaceutical composition which comprises both a compound of formula (I) (or a salt, solvate or prodrug thereof) and at least one other anti-proliferative agent selected from alkylating agents, antitumour antibiotics, antimetabolites, natural alkaloids and inhibitors of protein tyrosine kinases and/or serine/threonine kinases.

In particular, a therapeutically effective amount of each of the combination partners of the combination of the invention may be administered simultaneously or sequentially and in any order, and the combination partners may be administered separately or as a fixed combination.

For example, the method of preventing or treating proliferative diseases according to the invention may comprise: (i) administration of the first agent (a) in free or pharmaceutically acceptable salt form; and (ii) administration of an agent (b) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single (e.g., fixed) combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A physician of ordinary skill can readily determine and prescribe the effective amount of the single active ingredients required to treat the disease state.

Daily dosages for combination parties (a) and (b) will, of course, vary depending on a variety of factors, e.g., the compound chosen, the particular disease to be treated and the desired effect. In general, however, satisfactory results may be achieved on administration of agent (a) at daily dosage rates of about 0.03 to 5 mg/kg per day, particularly 0.1 to 5 mg/kg per day, e.g. 0.1 to 2.5 mg/kg per day, as a single dose or in divided doses. Combination partner (a) and agent (b) may be administered by any conventional route, in particular enterally, e.g., orally, e.g., in the form of tablets, capsules, drink solutions or parenterally, e.g., in the form of injectable solutions or suspensions. Suitable unit dosage forms for oral administration comprise from about 0.02 to 50 mg active ingredient, usually 0.1 to 30 mg, e.g. combination partner (a) or (b), together with one or more pharmaceutically acceptable diluents or carriers therefore.

Combination partner (b) may be administered to a human in a daily dosage range of 0.5 to 1000 mg. Suitable unit dosage forms for oral administration comprise from about 0.1 to 500 mg active ingredient, together with one or more pharmaceutically acceptable diluents or carriers therefore.

The pharmaceutical compositions for separate administration of combination partner (a) and agent (b) or for the administration in a fixed combination (i.e., a composition comprising both (a) and (b)) according to the invention, may be prepared in a manner known in the art and are those suitable for enteral, such as oral or rectal, and parenteral administration to mammals (warm-blooded animals), particularly humans, comprising a therapeutically effective amount of at least one pharmacologically active combination partner alone, e.g., as indicated above, or in combination with one or more pharmaceutically acceptable carriers or diluents, especially suitable for enteral or parenteral application.

Suitable pharmaceutical compositions contain, e.g., from about 0.1% to about 99.9%, preferably from about 1% to about 60%, of the active ingredient(s).

The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the subject. Compositions include those suitable for oral, rectal, nasal, topical (including buccal and sublingual), vaginal or parental (including subcutaneous, intramuscular, intravenous and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the invention.

Compositions for rectal administration may be presented as a suppository with a suitable base comprising, for example, cocoa butter, glycerin, gelatine or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage compositions are those containing a daily dose or unit, daily sub-dose, as herein above described, or an appropriate fraction thereof, of the active ingredient.

It should be understood that in addition to the active ingredients particularly mentioned above, the compositions of this invention may include other agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

The present invention also relates to a kit of parts comprising
(i) an amount of a compound of formula (I) or a pharmaceutically acceptable salt, solvate or prodrug thereof in a first unit dosage form; and
(ii) an amount of at least one other anti-proliferative selected from alkylating agents, antitumour antibiotics, antimetabolites, natural alkaloids and inhibitors of protein tyrosine kinases and/or serine/threonine kinases, in each case, where appropriate, a pharmaceutically acceptable salt thereof, in the form of one, two or three more separate unit dosage forms, optionally with instructions for use of (i) and (ii) components in combination for treating a proliferative disease.

Preferably the kit comprises a first unit dosage form of (i) and a second unit dosage form of one other anti-proliferative agent (ii).

The invention furthermore relates to a commercial package comprising the combination according to the present invention together with instructions for simultaneous, separate or sequential use.

In an embodiment, the (commercial) product is a commercial package comprising as active ingredients the combination according to the present invention (in the form of two or three or more separate units of the components as described above), together with instructions for simultaneous, separate or sequential use, of any combination thereof, in the treatment of the disease states as mentioned herein.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Synthetic Protocols

### Preparation of 2-Bromo-7-acetoxy-3-(3,4,5-trimethoxybenzoyl)-6-methoxybenzofuran

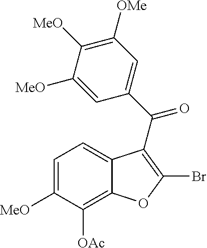 [SMILES: COc1cc(C(=O)c2c(Br)oc3c(OC(C)=O)c(OC)ccc23)cc(OC)c1OC]

### Step 1: 2-t-Butyldimethylsilyl-3-(t-butyldimethylsilyloxymethylene)-6-methoxy-7-isopropoxybenzofuran (Larock coupling)

A suspension of 2-isopropoxy-3-methoxy-5-iodophenol (4.41 mmol), 1-(tert-butyldimethylsilyl)-3-(tert-butyldimethylsilyloxy)propyne (1.5 g, 5.28 mmol), lithium chloride (189 mg, 4.45 mmol) and sodium carbonate (2.34 g, 22.08 mmol) in dry dimethylformamide (5 mL) at 100° C. was deoxygenated 4 times by evacuation and backfilling with nitrogen. Palladium acetate (135 mg, 0.60 mmol) was added and the reaction vessel was degassed twice with nitrogen. The reaction mixture was then stirred at this temperature for 4 hours (tic) and the solvent was removed by distillation under vacuum. The residue was dissolved in ethyl acetate (75 mL), stirred well, filtered and treated with triethylamine (5 mL). The solution was concentrated onto silica gel (10 g) and purified by flash chromatography (silica gel, eluent=hexane/diethyl ether/triethylamine; 95:5:1%) to afforded the title compound as a yellow oil (1.45 g, 96%); ¹H NMR (300 MHz, CDCl₃) δ 7.24 (d, 1H, J=8.45 Hz), 6.88 (d, 1H, J=8.47 Hz), 4.80 (s, 2H, CH₂), 4.73 (m, 1H), 3.88 (s, 3H, OMe), 1.36 (d, 6H, J=6.17 Hz), 0.94 (s, 9H), 0.92 (s, 9H), 0.35 (s, 6H), 0.12 (s, 6H).

### Step 2: 2-t-Butyldimethylsilyl-3-formyl-6-methoxy-7-isopropoxybenzofuran

To a solution of 2-t-butyldimethylsilyl-3-(t-butyldimethylsilyloxymethylene)-6-methoxy-7-isopropoxybenzofuran (2.69 mmol) in methanol (100 mL) was added concentrated hydrochloric acid (200 μL) and the reaction was stirred for 30 minutes (monitored by tic), quenched with triethylamine (2 mL) and the solvent removed by distillation under vacuum. The residue was dissolved in dichloromethane (20 mL), washed with water (10 mL), dried over magnesium sulfate, concentrated under vacuum and co-distilled with toluene (20 mL). The crude product was dissolved in dry dichloromethane (4 mL) and added to a stirred solution of Collin's reagent (chromium trioxide (1.01 g), pyridine (1.65 mL) in dry dichloromethane (30 mL)). The suspension was stirred for 10 minutes, filtered and the residue washed with diethyl ether (20 mL). The filtrate was concentrated onto silica (10 g) and purified by flash chromatography (silica gel, eluent=hexane/diethyl-ether/triethylamine (90:9:1) to afford the title compound as a light yellow oil (503 mg, 48%); ¹H NMR (300 MHz, CDCl₃) δ 10.25 (s, 1H, CHO), 7.79 (d, 1H, J=8.45 Hz), 6.98 (d, 1H, J=8.46 Hz), 4.65 (m, 1H), 3.89 (s, 3H, OMe), 1.35 (d, 6H, J=6.17 Hz), 0.97 (s, 9H), 0.45 (s, 6H).

### Step 3: 2-t-Butyldimethylsilyl-3-(3,4,5-trimethoxybenzoyl)-6-methoxy-7-isopropoxybenzofuran

To a stirred solution of 3,4,5-trimethoxyiodobenzene (377 mg, 1.27 mmol) in dry tetrahydrofuran (1 mL) at −78° C. under nitrogen was added n-butyllithium (795 μL, 1.59 mmol, 2M solution in cyclohexane) and the reaction mixture was stirred at this temperature for 40 minutes. After this time a solution of 2-t-butyldimethylsilyl-3-formyl-6-methoxy-7-isoproxybenzofuran (1.07 mmol) in dry tetrahydrofuran (1 mL) was added to the reaction dropwise via syringe pipette. The reaction mixture was stirred at −60° C. for 20 minutes and then allowed to warm to 0° C., stirred for 10 minutes, quenched with saturated ammonium chloride solution (2 mL) and diluted with ethyl acetate (20 mL). The organic layer was washed with water (10 mL), dried over magnesium sulfate and the solvent was removed under vacuum to give a residue that was co-distilled with toluene. The crude product (908 mg) was dissolved in dry tetrahydrofuran (10 mL) and treated with 2,3-dichloro-5,6-dicyano-1,4-benzoquinone (900 mg, 1.59 mmol) was added. The reaction mixture was stirred at room temperature for 16 hours (monitored by tic) and then loaded onto silica (10 g) and purified by flash chromatography (silica gel, eluent=hexane/diethyl ether/triethylamine, 90:9:1) to afford the title compound as a light yellow oil (498 mg, 69%); ¹H NMR (300 MHz, CDCl₃) δ 7.14 (s, 2H, benzoyl Hs), 6.81 (d, 1H, J=8.64 Hz), 6.77 (d, 1H, J=8.64 Hz) 4.74 (m, 1H), 3.93 (s, 3H, OMe), 3.86 (s, 3H, OMe), 3.78 (s, 6H, 2×OMe), 1.39 (d, 6H, J=6.14 Hz), 1.01 (s, 9H), 0.26 (s, 6H).

### Step 4: 2-(tert-butyldimethylsilyloxy)-7-acetoxy-3-(3,4,5-trimethoxybenzoyl)-6-methoxybenzofuran

To a stirred solution of 2-(t-butyldimethylsilyloxy)-7-isopropoxy-3-(3,4,5-trimethoxybenzoyl)-6-methoxy-benzofuran (160 mg, 0.31 mmol) in dry DCM (2 mL) at room temperature under nitrogen was added solid aluminium trichloride (83 mg, 0.62 mmol) and the reaction mixture was stirred for 15 minutes (monitored by tic). The reaction was quenched with a saturated solution of ammonium chloride, extracted with dichloromethane and dried over magnesium sulfate. The solvent was removed by distillation and residue was dried by azeotropic removal of water with toluene. The crude product was dissolved in pyridine (2 mL), acetic anhydride (1 mL) was added and reaction mixture was stirred for 2 hours at room temperature. The solvent was distilled under vacuum and the residue was loaded onto silica gel (1 g) and purified by column chromatography (silica gel, eluent, hexane:diethyl-ether; 80:20) (134 mg, 84%); ¹H NMR (300 MHz, CDCl₃) δ 7.14 (s, 2H, benzoyl Hs), 6.98 (d, 1H, J=8.72 Hz), 6.85 (d, 1H, J=8.72 Hz), 3.93 (s, 3H, OMe), 3.86 (s, 3H, OMe), 3.80 (s, 6H, 2×OMe), 2.41 (s, 3H), 0.99 (s, 9H), 0.25 (s, 6H).

### Step 5: 2-Bromo-7-acetoxy-3-(3,4,5-trimethoxybenzoyl)-6-methoxybenzofuran

To a stirred solution of 2-t-butyldimethylsilyl-7-acetoxy-3-(3,4,5-trimethoxybenzoyl)-6-methoxybenzofuran (120 mg, 0.44 mmol) in 1,2-dichloroethane (1 mL) at room temperature under nitrogen was added bromine (12 μl, 0.44 mmol) dropwise and the reaction mixture was stirred at this temperature for 10 minutes. After this time the reaction was quenched with saturated sodium thiosulfate solution, extracted with ethyl acetate (20 mL), dried over magnesium sulfate and the solvent removed by distillation under vacuum. The crude product was purified by silica gel column chromatography (eluent=Hexane:diethyl ether; 8:2-7:3) to afford the title compound as a colourless crystalline solid (91 mg, 81%); ¹H NMR (300 MHz, CDCl₃) δ 7.40 (d, 1H, J=8.70 Hz), 7.14 (s, 2H, benzoyl-Hs), 6.98 (d, 1H, J=8.75 Hz), 3.94 (s, 3H, OMe), 3.89 (s, 3H, OMe), 3.86 (s, 6H, 2×OMe), 2.43 (s, 3H); ¹³C NMR (75 MHz, CDCl₃) δ 187.95 (CO), 167.71, 152.75, 149.54, 147.49, 142.59, 131.92, 131.80, 123.91, 121.84, 119.89, 117.72, 109.89, 106.92, 60.69, 56.61, 56.00, 20.09.

### Example 1

### Preparation of 2-Methyl-7-hydroxy-3-(3,4,5-trimethoxybenzoyl)-6-methoxybenzofuran

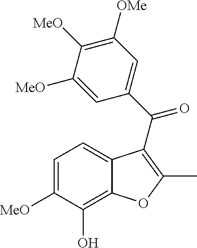 [SMILES: COc1cc(C(=O)c2c(C)oc3c(O)c(OC)ccc23)cc(OC)c1OC]

### Preparation A

To a stirred solution of 2-Bromo-7-acetoxy-3-(3,4,5-trimethoxybenzoyl)-6-methoxybenzofuran (20 mg, 0.042 mmol), methyl-boronic acid (40 mg, 0.67 mmol), in 1,4-dioxane (2 mL) at 90° C. was added tetrakis-triphenylphosphine palladium (11 mg, 0.01 mmol) followed by the addition of a solution of sodium bicarbonate (40 mg, 0.48 mmol) in distilled water (0.5 mL). The reaction mixture turned red after 5 minutes. After 2 hours (tic) the reaction mixture was brought to room temperature and was added saturated ammonium chloride (2 mL) and diluted with dichloromethane (20 mL). The organic layer was separated and washed with water, dried over magnesium sulfate and the solvent was removed by distillation under vacuum. The residue was purified by PTLC (eluent=Dichloromethane/Methanol, 1:1) to give the title compound (acetate cleaved during reaction) as a fluffy white solid; (3 mg, 19%).

### Preparation B (Negishi Coupling)

To a stirred solution of zinc-bromide (592 mg, 2.63 mmol) in dry THF(1.5 mL) at 0° C. was added the solution of methyl lithium (1.6 M solution in diethyl-ether, 2.6 mL, 4.15 mmol) and the reaction mixture was stirred for 2 hours. Solid 2-bromo-7-acetoxy-3-(3,4,5-trimethoxybenzoyl)-6-methoxy-benzofuran (300 mg, 0.63 mmol) was added and the ether was removed under vacuum and to the rest suspension was added dichlorobis(triphenylphosphine)palladium catalyst (21 mg) and catalytic amount of copper (I) iodide. The reaction mixture was stirred at room temperature for 36 hours (monitored by tic), quenched with saturated ammonium chloride solution and extracted with dichloromethane (10 mL), dried over magnesium sulfate and solvent distilled under vacuum and the product was purified by silica gel column (eluent=hexane/ethyl acetate; 8:2). The product was crystallized in methanol (106 mg, 46%); ¹H NMR (300 MHz, CDCl₃) δ 7.09 (s, 2H, benzoyl Hs), 6.93 (d, 1H, J=8.54 Hz), 6.83 (d, 1H, J=8.56 Hz), 5.70 (bs, 1H, OH), 3.93 (s, 3H, OMe), 3.92 (s, 3H, OMe), 3.83 (s, 6H, 2×OMe), 2.54 (s, 3H, 2-Me)

### Example 2

### Preparation of Disodium 6-methoxy-2-methyl-3-(3,4,5-trimethoxybenzoyl)benzofuran-7-yl phosphate

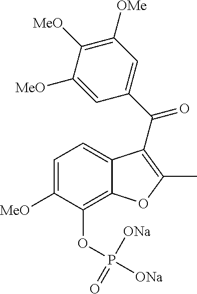

### Step 1: Dibenzyl 6-methoxy-2-methyl-3-(3,4,5-trimethoxybenzoyl)benzofuran-7-yl phosphate

To a mixture of 0.081 g (0.22 mmol) of (7-hydroxy-6-methoxy-2-methylbenzofuran-3-yl)(3,4,5-trimethoxyphenyl)methanone, 0.086 g (0.261 mmol) of carbon tetrabromide and 0.063 ml (0.283 mmol) of dibenzylphosphite in 2.5 ml of anhydrous acetonitrile 0.046 ml of anhydrous triethylamine was added dropwise at 0° C. under nitrogen atmosphere. The resulting mixture was stirred for 2 h at room temperature, then diluted to 20 ml with ethyl acetate, washed with water brine, dried over anhydrous magnesium sulfate, filtered off and evaporated to dryness under reduced pressure. The residue was purified by flash column chromatography (dichloromethane/ethyl acetate, 9:1) to give the title compound as a colorless foam (0.13 g, 94%); ¹H NMR (CDCl₃) δ 2.42 (s, 3H, Me-2); 3.83 (s, 1H, OMe); 3.93 (s, 3H, OMe); 5.33 (m, 4H, CH₂Ph); 6.89 (d, CH aromatic, J=8.7 Hz); 7.21 (dd, 1H, CH aromatic, J=8.72 Hz; J=1.2 Hz); 7.08 (s, 2H, CH aromatic); 7.29-7.43 (m, 10H, CH aromatic).

### Step 2: Disodium 6-methoxy-2-methyl-3-(3,4,5-trimethoxybenzoyl)benzofuran-7-yl phosphate

To a stirred solution of 0.122 g (0.193 mmol) of the product from Step 1 in 1 ml of anhydrous acetonitrile 0.075 ml (0.58 mmol) of bromotrimethylsilane was added at −5° C. under nitrogen atmosphere. The resulting mixture was stirred for 1 h at 0° C., then evaporated to dryness in vacuo. The residue was diluted to 5 ml with anhydrous methanol and pH of the solution was brought up about 10 by the addition of sodium methoxide. After evaporation of the resulting mixture under reduced pressure the solid residue was washed with anhydrous isopropanol (4×1.5 ml) and anhydrous ethanol (3×1.5 ml) and dried under vacuum to give 0.062 g (65% yield) of title compound as an colorless solid; ¹H NMR (D₂O) δ 2.37 (s, 3H, Me-2); 3.76 (s, 6H, OMe); 3.79 (s, 3H, OMe); 3.82 (s, 3H, OMe); 4.66 (s, H₂O); 6.93 (d, 1H, CH aromatic, J=8.6 Hz); 7.04 (d, 1H, CH aromatic, J=8.6 Hz); 7.10 (s, 2H, CH aromatic).

### Biological Data

### (A) (i) In Vitro Studies for Combination Partner (a)

TABLE 1
In Vitro Data for Compounds: These are the results for growth inhibition
studies of compounds using the Sulforhodamine B (SRB) or Systmex cell counting (CC)
assays. IC₅₀ is the concentration required to inhibit net cell growth by 50%. Entries 1-4
are provided for comparison, entry 5 is a compound of the invention (combination partner
(a)).

| | | | Cancer | |
|---|---|---|---|---|
| | | | cell | HUVECs^{c} |
| | | | line^{a}: | Tum: IC₅₀, nM |
| | Example/ | | IC₅₀, | Norm: IC₅₀, |
| Entry | Comparator | Structure | nM | nM |
| 1. | Comparator A | 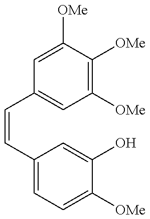 [SMILES: COc1ccc(/C=C\c2cc(OC)c(OC)c(OC)c2)cc1O] | 5 | Turn: 1-10 Norm: 1-10 |
| 2. | Comparator B | 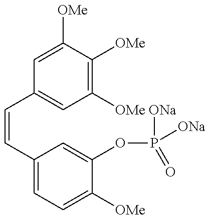 | 5 | Turn: 1-10 Norm: 1-10 |
| 3. | Comparator C Example 5 | 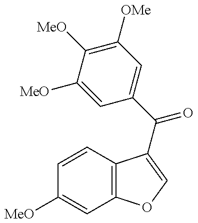 [SMILES: COc1ccc2c(C(=O)c3cc(OC)c(OC)c(OC)c3)coc2c1] | 55 | Turn: 10-100 Norm: 10-100 |
| 4. | Comparator D Example 3 | 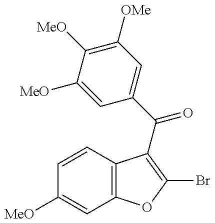 [SMILES: COc1ccc2c(C(=O)c3cc(OC)c(OC)c(OC)c3)c(Br)oc2c1] | 500 | Turn: 100-1000 Norm: 100-1000 |
| 5. | Example 1 | 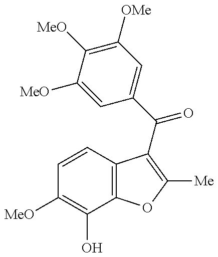 [SMILES: COc1cc(C(=O)c2c(C)oc3c(O)c(OC)ccc23)cc(OC)c1OC] | 2.0 | Turn: 0.1-1 Norm: 10-100 |

^{a}Unless otherwise stated the cancer cell line is MCF-7. For description of method of MCF-7 inhibition see: Verdier-Pinard, P et al.Mol. Pharmacol 1998, 53, 62-76
^{c}Human umbilical vein endothelial cells (HUVECs) tumour type activated endothelial cells (Turn) and normal quiescent type endothelial cells (Norm).

### General Description of Biological Experiments

Proliferation Assay—Quiescent endothelium: Human umbilical vein endothelial cells (CC-2519, Clonetics) were plated at 15000 cells/well in EBM2 (CC-3156, Clonetics)+0.5% FBS (CC-4101A, Clonetics)+GA-1000 (CC-4381A, Clonetics) in a 96 well plate in triplicate. Cells were cultured overnight at 37° C. 5% CO₂. Medium was subsequently replaced with fresh medium including the compound or negative control. Cells were cultured for a period of 48 hrs. An MTT assay was performed to measure changes in cell numbers. Briefly, 20 μl of MTT reagent was added to cells containing 100 μl of EBM2+0.5% FBS and incubated at 37° C. for 2 hours. Absorbance was measured at 492 nm.

Proliferation Assay—activated endothelium: Human umbilical vein endothelial cells (CC-2519, Clonetics) were plated at 2500 cells/well in EGM2 (CC-3162, Clonetics) in a 96 well plate in triplicate. Cells were cultured overnight at 37° C. 5% CO₂. Medium was subsequently replaced with fresh medium including the compound or negative control. Cells were cultured for a period of 48 hrs. An MTT assay was performed to measure changes in cell numbers. Briefly, 20 μl of MTT reagent was added to cells containing 100 μl of EGM2 and incubated at 37° C. for 2 hours. Absorbance was measured at 492 nm.

### (ii) In Vivo Studies for Combination Partner (a)

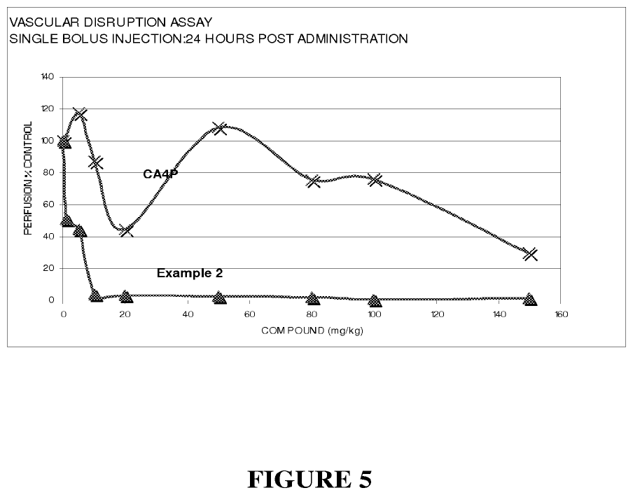

Vascular Disruption Assay: Female athymic BALB/c-nu/nu mice (nude mice) were used for this study. Mice were between 6-8 weeks old and were purchased from the Animal Resource Centre, Perth, Western Australia and allowed to acclimatize for a couple of days. All the animals were housed under pathogen-free conditions and cared for in accordance with Flinders University of South Australia and NH&MRC guidelines and the Australian Code of Practice for the care and use of animals for scientific purposes. The human breast cancer MDA MB 231 was grown as orthotopic xenografts in the mammary fat pad of nude mice. Each mouse was injected with 2×10⁶ cells in 50 μl Dulbecco's PBS subcutaneously just above the mammary fat pad, below the right forward limb. Tumours were selected for treatment when they reached a diameter of 100-150 mm³ (3 weeks after implantation). The test compound (Example 2) was dissolved in saline solution and injected intravenously at concentrations ranging from 150 mg/kg-1 mg/kg in a total volume of 400 ul. Tumour bearing animals were injected intravenously with 10 mg/kg Hoechst 33342, 24 hours after the injection of the test compound. Animals were euthanised 1 minute after the Hoechst 33342 injection. Tumours were recovered for histochemical analysis. Tumour perfusion analysis was performed by assessing the amount of Hoechst 33342 staining across an entire tumour cross-section. 10 micron sections of frozen tumour biopsies were viewed under an ultraviolet light filter. Using a 4× objective lens, 8-bit monochromatic images were captured in succession, representing the total area of the tumour section. Composite images of the total tumour section were generated by overlaying common areas of the monochromatic images. Hematoxylin and Eosin-Y staining of the same tumour section was performed to identify non-tumour regions. Non-tumour regions were mapped on Hoechst 33342 composite images and excluded from the quantitation analysis. Quantitation was performed by measuring the pixel area of Hoechst 33342 staining and the total pixel area of the tumour region. Perfusion was expressed as a percentage of Hoechst 33342 stained area to total tumour area (see FIG. 5).

Tumour Growth Inhibition: Balb/c nu/nu mice bearing MDA-MB-231 solid orthotopic tumours were treated with compound Example 2 at 40 mg/kg. Animals were i.v. dosed with a total of two cycles of Example 2 treatment. Each cycle was dosing on days and 8 followed by a three week no-dosing period. Tumour growth represented as a ratio to initial tumour volume is shown over a total of 72-days.

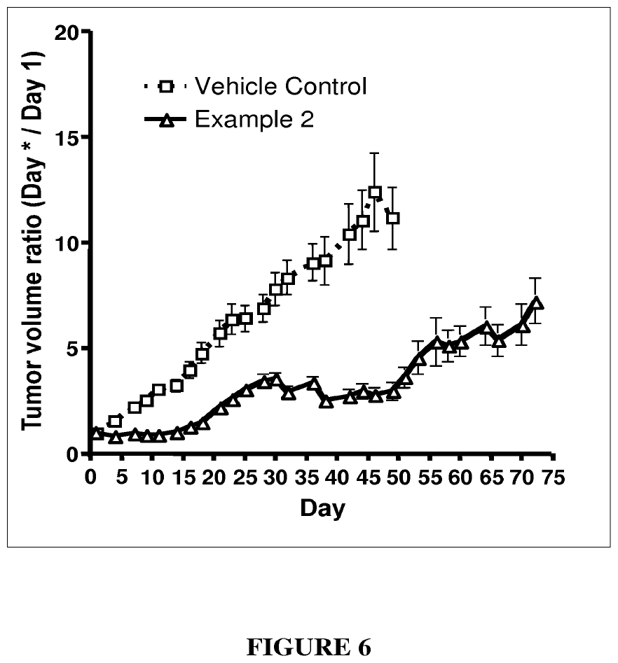

Tumour growth as well as animal health were monitored for up to 72 days post-day 1 of treatment. The results seen in this experiment (see FIG. 6) clearly show tumour growth inhibition in animals treated with two cycles of Example 2. Significant differences in tumour growth between Example 2 treated (n=64) and vehicle treated (n=20) animals were observed as early as day 4 (p<0.001; unpaired t-test; Prism® analysis) through to Day 70.

### (B) (i) In Vitro Studies for Combinations of (a) and (b)

### Combination Index Values

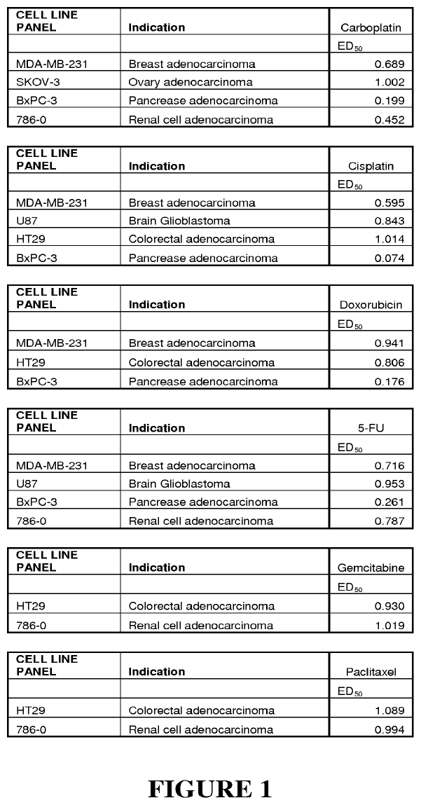

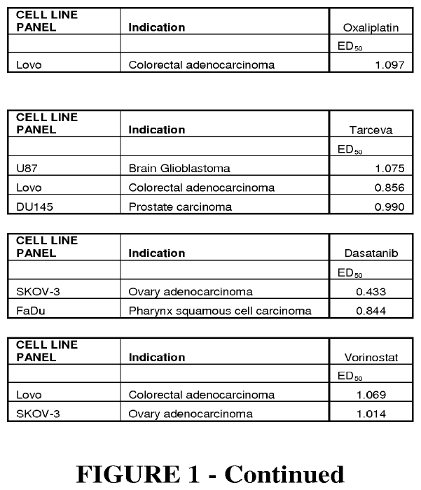

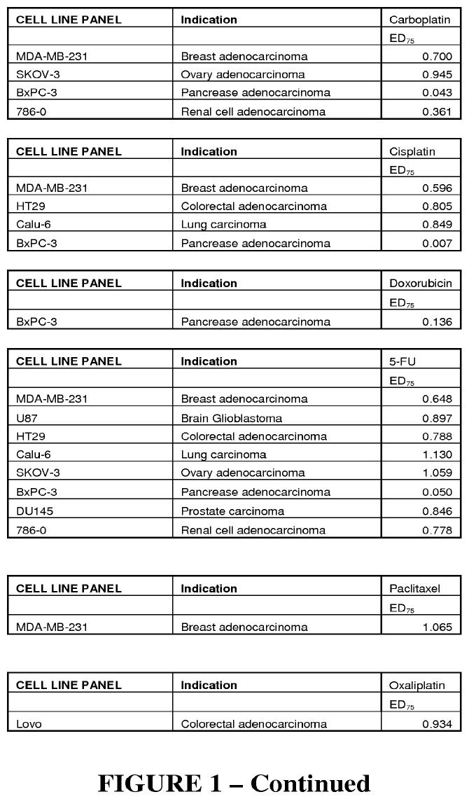

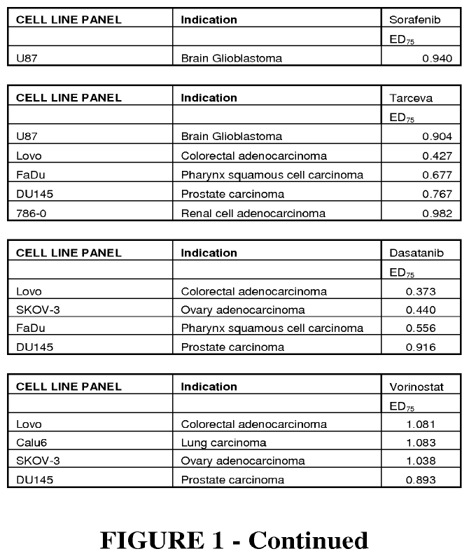

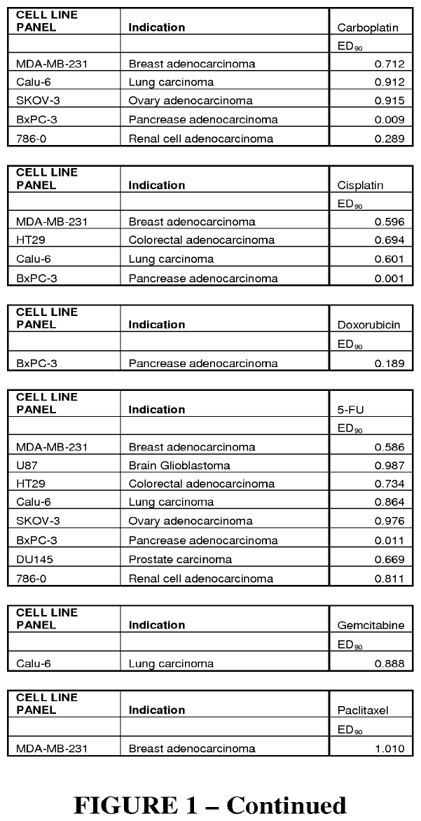

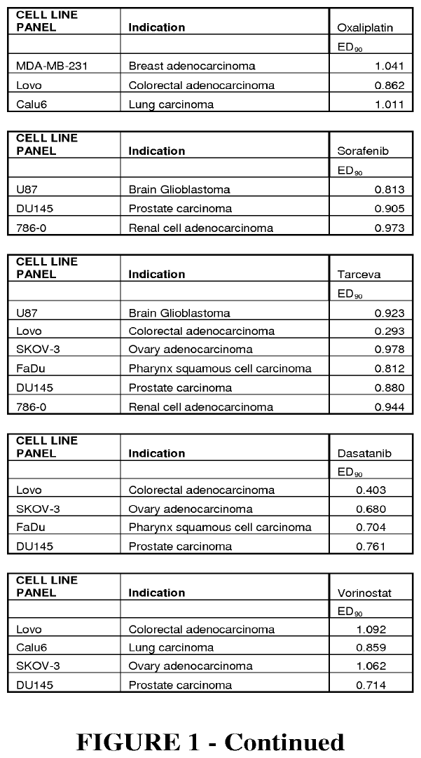

Human cancer cell lines (list cell lines: MDA-MB-231, U87, HT29, Calu-6, SKOV-3, BxPC-3, DU145, 786-0, Lovo, FaDu) were used to evaluate combination treatments in vitro. Analysis was based on measurements of in vitro cell proliferation in the presence of the compounds under evaluation. Cells were seeded at an average of 500-2000 cells/well in 96 well plates allowed to adhere overnight before addition of the test compounds. Cell proliferation was assessed after 48-72 hrs of culture in the presence of the test substances. Cells were treated with a combination of Example 1 and the compound under evaluation, or with each of these agents alone. Proliferation measurements were carried out by a tetrazolium-based colorimetric assay (MTT). Metabolically active cells were measured using CellTiter 96® Aqueous One Solution (Promega Corp. Madison Wis., USA) according to the manufacturers instructions and absorbance readings taken at 492 nm. Absorbance readings for each compound concentration were normalized to corresponding vehicle control cultures. A sigmoidal dose response curve was fitted to the data, and the concentration at which proliferation decreased by 50% was calculated using Graph Pad Prism 4 software (San Diego, USA). ED50, ED75 and ED90 data derived from these evaluations were analyzed using the quantitative software CaluSyn to determine the combination index (CI) (Chou 2006 Theoretical Basis, Experimental Design and Computerised Simulation of Synergism and Antagonism in Drug Combination Studies, Pharmacological Reviews 58 (3): 621-681). Based on their CI, compounds were classified as antagonistic, additive or synergistic combination partners for Example 1. The results are tabulated in FIG. 1.

CalcuSyn is a dose effect analyzer program for single and multiple drugs. It uses the Median Effect method to quantify the effects of drug combinations to determine whether they give greater effects together that expected from a simple summation of their individual effects. Data was processed for individual drugs and for constant ratio combinations. The program automatically graphs the data and produces reports of summary statistics for all drugs and detailed analysis of drug interactions including the Combination Index and EDx (effective dose).

Cell culture and cell lines. Cancer cell lines included Calu-6, DU145, U87-MG, BxPc-3, HT29, Sk-OV-3, 786-0, LoVo (ATCC, Manassas, Va., USA), FaDu (kind gift from Peter Mac, Melbourne, AU) and MDA-MB-231 (kind gift from WCH, Adelaide, AU).

Calu-6, DU145 and U87-MG cells were cultured in MEM media (Gibco®) with 10% FCS, 2 mM penicillin-streptomycin-glutamine (Gibco®), 10 mM Hepes buffered solution (Gibco®), 1 mM sodium pyruvate solution (Gibco®) and 0.1 mM non-essential amino acids solution (Gibco®). FaDu cells were cultured in RPMI 1640 media (Gibco®) with 10% FCS and 10 mM Hepes buffered solution. Bx-PC-3 cells were cultured in RPMI 1640 media with 10% FCS, 2 mM penicillin-streptomycin-glutamine and 1 mM sodium pyruvate solution. MDA-MB-231 cells were cultured in RPMI 1640 media with 10% FCS, 2 mM penicillin-streptomycin-glutamine and 10 mM Hepes buffered solution. HT29 cells were cultured in MEM media with 10% FCS and 2 mM penicillin-streptomycin-glutamine. SK-OV-3 cells were cultured in DMEM/F12 (Gibco®) media with 10% FCS, 2 mM penicillin-streptomycin-glutamine and 10 mM Hepes buffered solution. 786-0 cells were cultured in in RPMI 1640 media with 10% FCS, 2 mM penicillin-streptomycin-glutamine, 10 mM Hepes buffered solution and 1 mM sodium pyruvate solution and 0.1 mM non-essential amino acids solution. Lovo cells were cultured in F12K (Gibco®) media with 10% FCS, 2 mM penicillin-streptomycin-glutamine and 10 mM Hepes buffered solution.

All cells were cultured in a humidified incubator at 37° C. with 5% CO₂.

### (B) (ii) In Vivo Studies for Combinations of (a) and (b)

### (a) Example 2 Treatment in Combination with Cisplatin (Combination Partner (b))

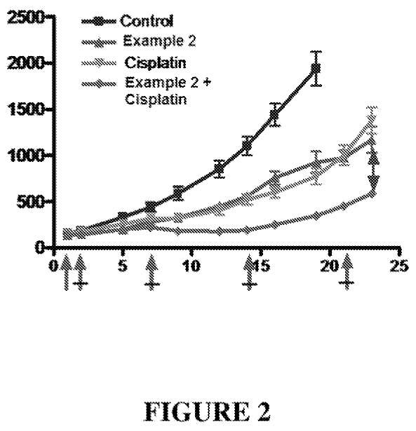

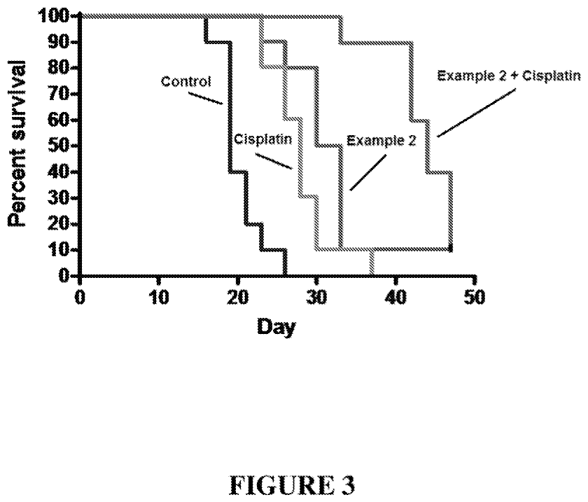

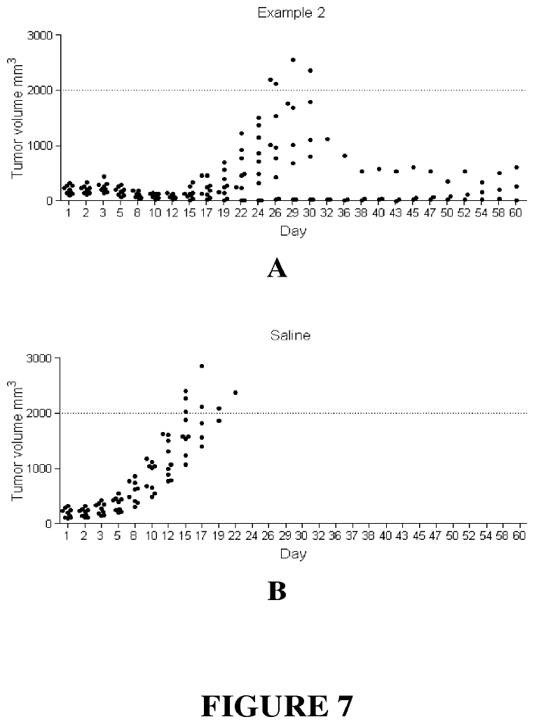

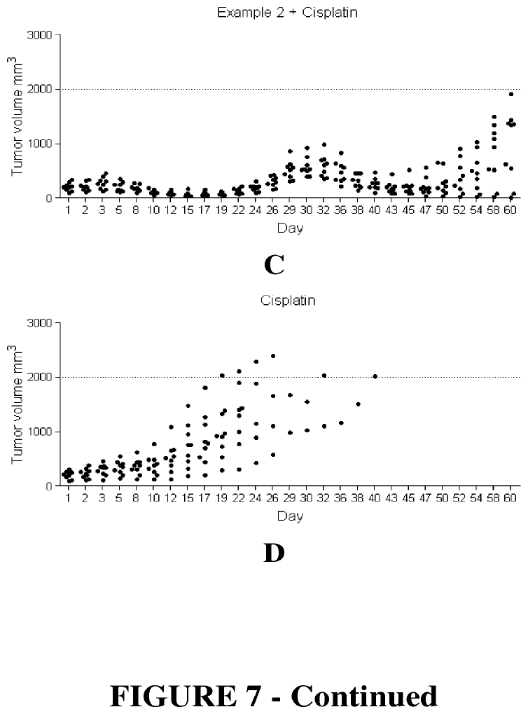

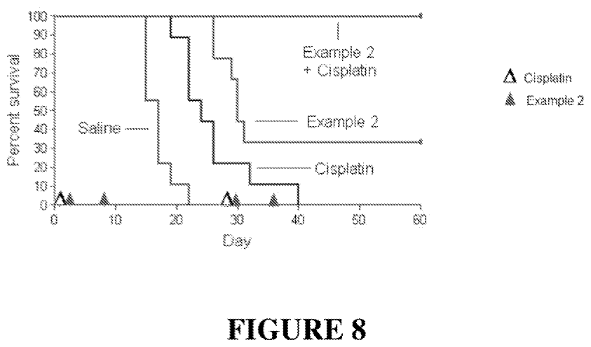

(i) Balb/c nu/nu mice were inoculated with 2×10⁶ Calu-6 cells (human lung carcinoma line) subcutaneously. Once mean tumour size had reached 150 mm³ mice were treated with cisplatin on day 1 at 4 mg/kg, weekly treatment of Example 2 at 10 mg/kg starting on day 2 or a combination of both drugs (n=10/group). Tumour volume measurement were taken throughout the treatment period and statistical analysis performed on complete groups. Survival was assessed throughout the trial and is shown below. Average weight loss in combined treatment group did not exceed acceptable levels. The results are illustrated in FIG. 2 and FIG. 3.
(ii) Female BALB/c nu/nu mice were subcutaneously inoculated with the human cell line Calu-6 derived from a lung cancer to establish solid tumors. Tumors were grown to an average size of 200 mm³ before commencing treatment. Tumor volume (in cubic millimeters) was measured two to three times per week. Animals were iv dosed Example 2 (Solution for Injection) at 32 mg/kg* on Day 2 and Day 9 of a 28 day cycle and/or ip dosed Cisplatin at 4 mg/kg on Day 1 of a 28 Day cycle (n=9/treatment). Tumour growth (FIG. 7) and survival (FIG. 8) were monitored over the course of the 60 day study. Reduced tumor volume and increased survival is seen in the combination therapy group (Example 2+Cisplatin) compared to monotherapies alone (Example 2 or Cisplatin) or saline vehicle control.
   Dosage values of 32 mg/kg Example 2 (Solution for Injection) take into account the quantity of active material in the test material (excluding water and sodium) and is equivalent to a dose of 40 mg/kg Example 2 (including water and sodium).

### (b) Example 2 Treatment in Combination with 5-Fluorouracil or Doxorubicin (Combination Partner (b))

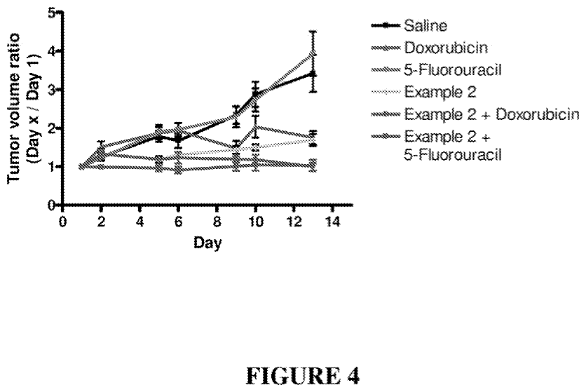

Balb/c nu/nu mice were inoculated with 2×10⁶ MDA-MB-231 cells (human breast carcinoma line) subcutaneously. Once mean tumour size had reached 50-100 mm³ mice were treated with Example 2 (10 mg/kg) as a single i.v. dose every 96 hrs followed on the next day by either a saline injection or injection of the chemotherapeutics Doxorubicin (5 mg/kg) or 5-Fluorouracil (50 mg/kg). Tumour volume measurements were taken throughout the treatment period and are expressed as a ratio relative to starting volume on day 1. Results are illustrated in FIG. 4.

### (c) Example 2 Treatment in Combination with Gemcitabine (Combination Partner (b))

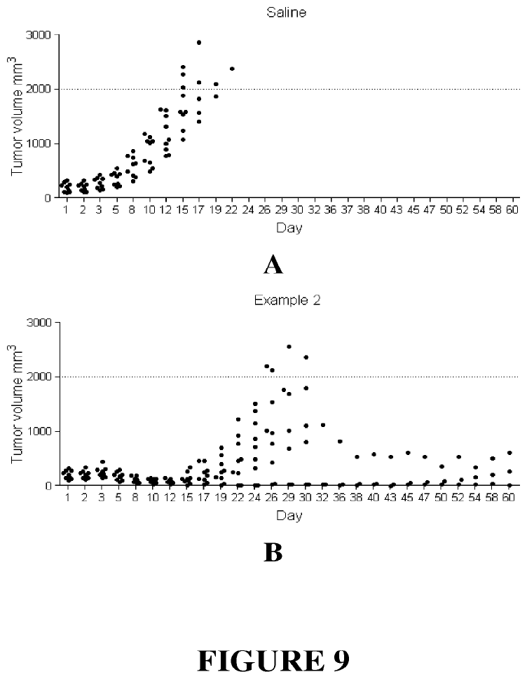

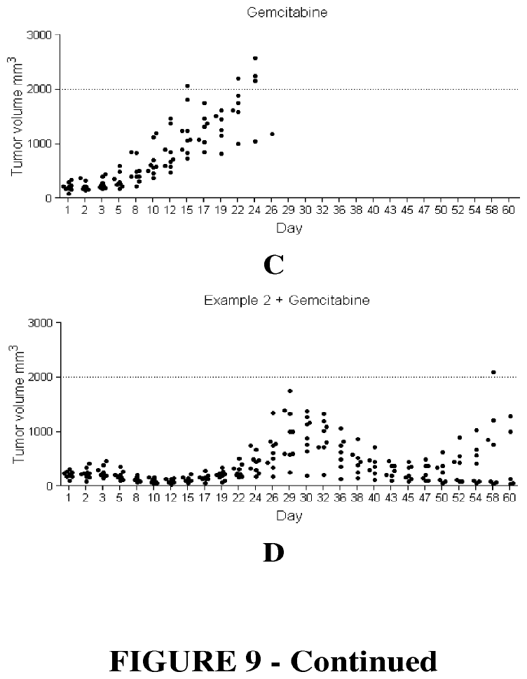

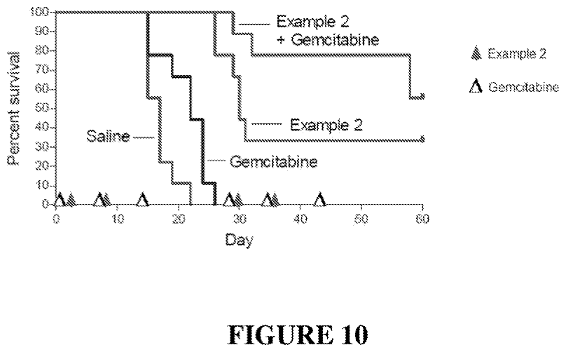

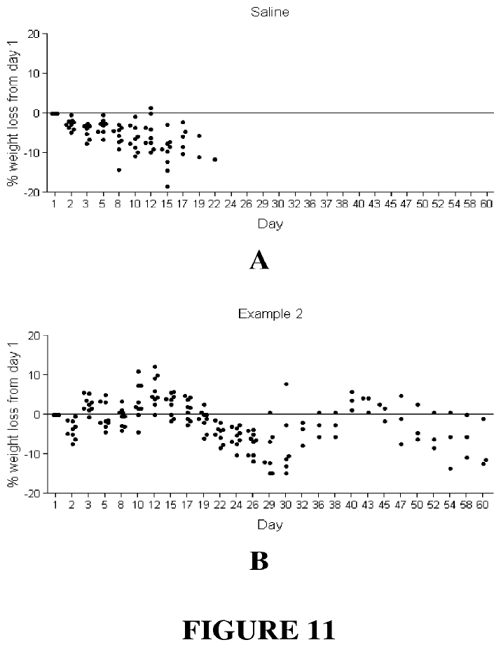

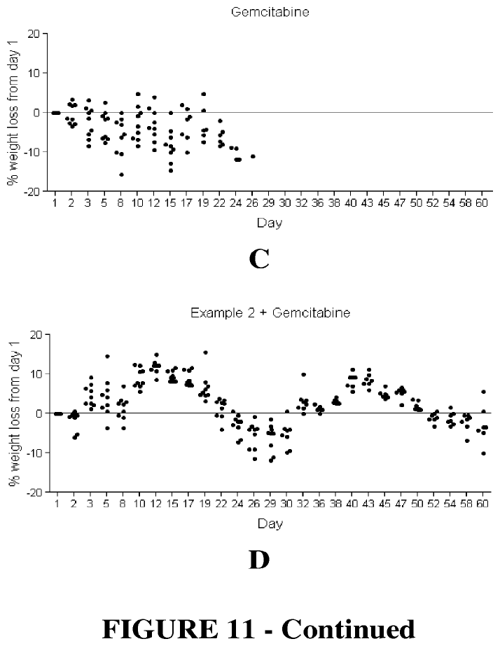

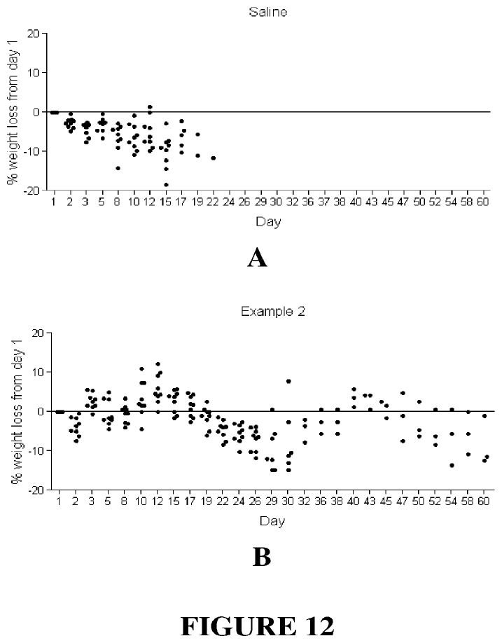

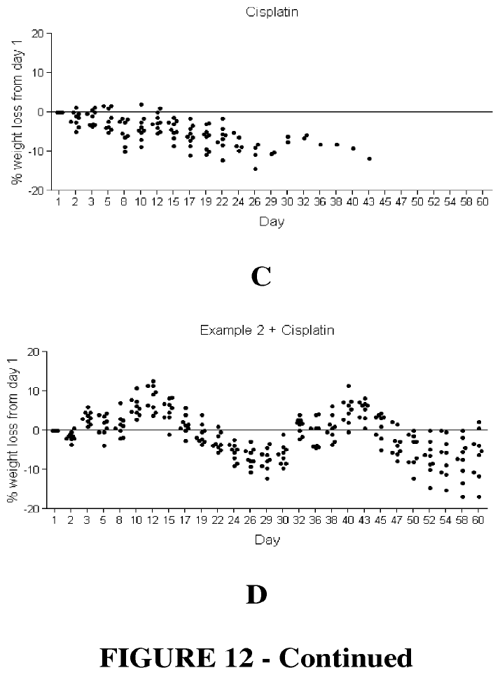

Female BALB/c nu/nu mice were subcutaneously inoculated with the human cell line Calu-6 derived from a lung cancer to establish solid tumors. Tumors were grown to an average size of 200 mm³ before commencing treatment. Tumor volume (in cubic millimeters) was measured two to three times per week. Animals were iv dosed Example 2 at 32 mg/kg*(Solution for Injection) on Day 2 and Day 9 of 28 day cycle and/or ip dosed Gemcitabine at 50 mg/kg on Day 1, Day 8 and Day 15 of 28 Day cycle (n=9/treatment). Tumour growth (FIG. 9) and survival (FIG. 10) were monitored over the course of the 60 day study. Reduced tumor volume and increased survival is seen in the combination therapy group (Example 2+Gemcitabine) compared to monotherapies alone (Example 2 or Gemcitabine) or saline vehicle control.
Dosage values of 32 mg/kg Example 2 (Solution for Injection) take into account the quantity of active material in the test material (excluding water and sodium) and is equivalent to a dose of 40 mg/kg Example 2 (including water and sodium).

1. A method for treating a proliferative disease including the step of administering to a patient in need thereof (a) a compound of formula (I) or a pharmaceutically acceptable salt, solvate or prodrug thereof; 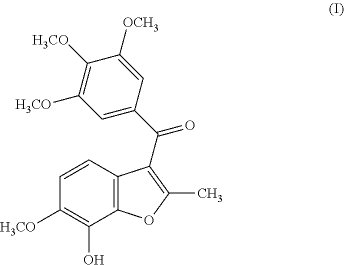 [SMILES: COc1cc(C(=O)c2c(C)oc3c(O)c(OC)ccc23)cc(OC)c1OC]
and (b) at least one other anti-proliferative agent selected from the group consisting of: carboplatin, cisplatin, and gemcitabine,
wherein the proliferative disease is a tumour and wherein (a) is administered to said patient intravenously from about 8.4 to about 16.0 mg/m².

2. The method according to claim 1, wherein the compound of formula (I) is in a prodrug form.

3. The method according to claim 2, wherein the prodrug form is represented by the formula

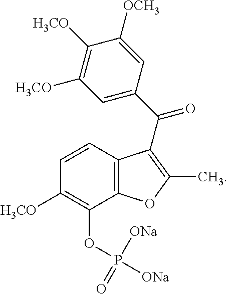

4. The method according to claim 1, wherein the tumour is selected from the group consisting of: breast carcinoma, brain glioblastoma, colorectal carcinoma, lung carcinoma, ovary carcinoma, pancreas carcinoma, prostate carcinoma, renal cell carcinoma, and pharynx squamous cell carcinoma.

5. The method according to claim 4 wherein (b) is carboplatin and the tumour is ovary carcinoma.

6. The method according to claim 4 wherein (b) is gemcitabine and the tumour is lung carcinoma.

7. The method according to claim 1, wherein (a) and (b) are separately administered as a single dose within 48 hours of each other.

8. The method according to claim 1, wherein (a) and (b) are separately administered as a single dose within 48 hours of each other and (a) is administered again at least one further time after at least about 96 hours from the first dose.

9. The method according to claim 1, wherein (a) and (b) are separately administered as a single dose within 48 hours of each other and (a) is administered again at least two further times after at least about 96 hours from the first dose.

10. The method according to claim 1, wherein (a) and (b) are separately administered as a single dose within 48 hours of each other and (a) is administered again at least two further times after at least about 96 hours from the first dose in a 28 day cycle.
